(19) United States
(12) Patent Application Publication
(10) Pub. No.: US 2018/0362418 A1
(43) Pub. Date: Dec. 20, 2018

(54) CONVERSION OF METHANE TO ETHYLENE COMPRISING INTEGRATION WITH THE IN-SITU ETHANE CRACKING AND DIRECT CONVERSION OF CO2 BYPRODUCT TO METHANOL

(71) Applicant: SABIC Global Technologies B.V., Bergen op Zoom (NL)

(72) Inventors: Aghaddin MAMEDOV, Sugar Land, TX (US); David WEST, Sugar Land, TX (US); Wugeng LIANG, Sugar Land, TX (US); Sagar SARSANI, Sugar Land, TX (US)

(73) Assignee: SABIC Global Technologies B.V., Bergen op Zoom (NL)

(21) Appl. No.: 16/060,991

(22) PCT Filed: Dec. 7, 2016
(86) PCT No.: PCT/IB2016/057402

§ 371 Date: Jun. 11, 2018

Related U.S. Application Data
(60) Provisional application No. 62/266,913, filed on Dec. 14, 2015.

(51) Int. Cl. C07C 2/84; C07C 5/42; B01J 23/887; C01B 3/38; C07C 27/06

(52) U.S. Cl. CPC C07C 2/84; C07C 5/42; C07C 31/04; C01B 3/38; C07C 27/06; B01J 23/8873

(57) ABSTRACT

Methods and catalysts for producing ethylene and methanol from natural gas are presented. Methods include integration of oxidative conversion of methane to ethane, ethane in situ thermal cracking using the thermal heat generated thereby and direct hydrogenation of byproducts to methanol or oxidative CO₂ autothermal reforming of methane to syngas.

### CROSS-REFERENCE TO RELATED APPLICATIONS

This application claims priority to and the benefit of U.S. Provisional Application No. 62/266,913, filed Dec. 14, 2015. The contents of the referenced application are incorporated into the present application by reference.

### FIELD

The presently disclosed subject matter relates to methods and systems for conversion of natural gas to ethylene and methanol.

### BACKGROUND

Ethylene can be used for production of bulk-chemicals, e.g., poly-ethylene and ethyleneoxide. Oxidative coupling of methane (OCM) can be used for the industrial production of hydrocarbons, e.g., ethylene, as shown below:

2CH₄+O₂→C₂H₄+2H₂O

2CH₄+½O₂→C₂H₆+H₂O

CH₄+1.5O₂→CO+2H₂O

CH₄+2O₂→CO₂+2H₂O (1)

However, one drawback of the OCM approach can include low ethylene yield. Low concentration of ethylene (low ethylene yield) produced from OCM can be a result of the highly exothermic reaction. The heat of the reaction can lead to an increase of catalyst bed temperature and heat runaway. This decreases selectivity for C₂ products. Additionally, carbon dioxide is usually released into the atmosphere as an environmentally damaging gas. Therefore, there remains a need in the art for methods of utilizing the heat generated by oxidative coupling of methane and increasing product selectivity.

### SUMMARY OF THE DISCLOSED SUBJECT MATTER

The presently disclosed subject matter provides processes for preparing ethylene from natural gas, including combining methane and oxygen gas in a reactor zone to undergo oxidative conversion to form produced ethylene, carbon dioxide, water, and heat. Example processes can further include providing ethane to a post-reactor zone. The process can also include cracking the ethane using the heat produced by the oxidative conversion to form ethylene; and contacting the produced carbon dioxide with a first catalyst to generate methanol.

In certain embodiments, the combining further includes contacting methane and oxygen gas with a second catalyst in the reactor zone. In certain embodiments, the second catalyst is 10% Na-15% Mn—O/SiO₂.

In certain embodiments, the first catalyst is CuO—ZnO—Cr₂O₃—Al₂O₃.

In certain embodiments, the first catalyst is 69.3% CuO-27.4% ZnO-4.24% Cr₂O₃-3.97% Al₂O₃. In other embodiments, the first catalyst is CuO—ZnO—Al₂O₃. In certain embodiments, the first catalyst is 44.26% CuO-36.44% ZnO-11.68% Al₂O₃. In further embodiments, the first catalyst is 55.2% CuO-24.9% ZnO-19.83% ZrO₂.

In certain embodiments, the contacting can include a pressure of from about 250 psi to about 900 psi, or from about 750 psi to about 800 psi.

In certain embodiments, the combining can include a temperature from about 750° C. to about 850° C. In certain embodiments, the temperature is about 830° C., about 740° C., or about 720° C.

In certain embodiments, the contacting can include a temperature from about 200° C. to about 300° C. for generation of methanol. In certain embodiments, the temperature is about 250° C.

In certain embodiments, ethylene selectivity is from about 10 to about 75% mol. In certain embodiments, the selectivity is about 13.5%, 44.2%, or 63.5% mol.

The presently disclosed subject matter also provides techniques for preparing ethylene from natural gas, which can include combining methane and oxygen gas in a reactor zone to undergo oxidative conversion to form produced ethylene, carbon dioxide, water, and heat. The process can further include providing ethane to a post-reactor zone. The process can also include cracking the ethane using the heat produced by the oxidative conversion to form ethylene, and contacting the produced carbon dioxide with a catalyst to generate syngas.

In certain embodiments, the catalyst for formation of syngas is 3% Ni/La₂O₃.

In certain embodiments, the combining further comprises O₂ and N₂. In other embodiments, the process includes 28.4% CH₄, 17.4% CO₂, 11% O₂, and 42.8% N₂.

### BRIEF DESCRIPTION OF THE DRAWINGS

FIG. 1 is a schematic representation of one exemplary system of the presently disclosed subject matter.

FIG. 2 depicts a schematic representation of one exemplary method of the presently disclosed subject matter.

FIG. 3 is a schematic representation of one exemplary system of the presently disclosed subject matter.

### DETAILED DESCRIPTION

The presently disclosed subject matter provides systems and methods for conversion of natural gas to ethylene via integration of three processes: 1) oxidative conversion of methane to ethane, 2) ethane in situ thermal cracking using the thermal heat generated in process 1), and 3) direct hydrogenation of byproducts to methanol or oxidative CO₂ autothermal reforming of methane to syngas. The total reaction of the integrated processes can be represented by the following equation:

3CH₄+3C₂H₆+3O₂→4C₂H₄+CH₃OH+5H₂O (2)

In certain embodiments, the presently disclosed subject matter is directed to a system that includes at least two reactors for the production of ethylene and methanol from a natural gas stream. In certain embodiments, the presently disclosed subject matter is directed to a system that includes an oxidative coupling of methane (OCM) reactor coupled to a separation unit, coupled to a hydrogenation reactor for production of methanol and ethylene.

“Coupled” as used herein refers to the connection of a system component to another system component by any means known in the art. The type of coupling used to connect two or more system components can depend on the scale and operability of the system. For example, and not by way of limitation, coupling of two or more components of a system can include one or more transfer lines, joints, valves, fitting, coupling or sealing elements. Non-limiting examples of joints include threaded joints, soldered joints, welded joints, compression joints and mechanical joints. Non-limiting examples of fittings include coupling fittings, reducing coupling fittings, union fittings, tee fittings, cross fittings and flange fittings. Non-limiting examples of valves include gate valves, globe valves, ball valves, butterfly valves and check valves.

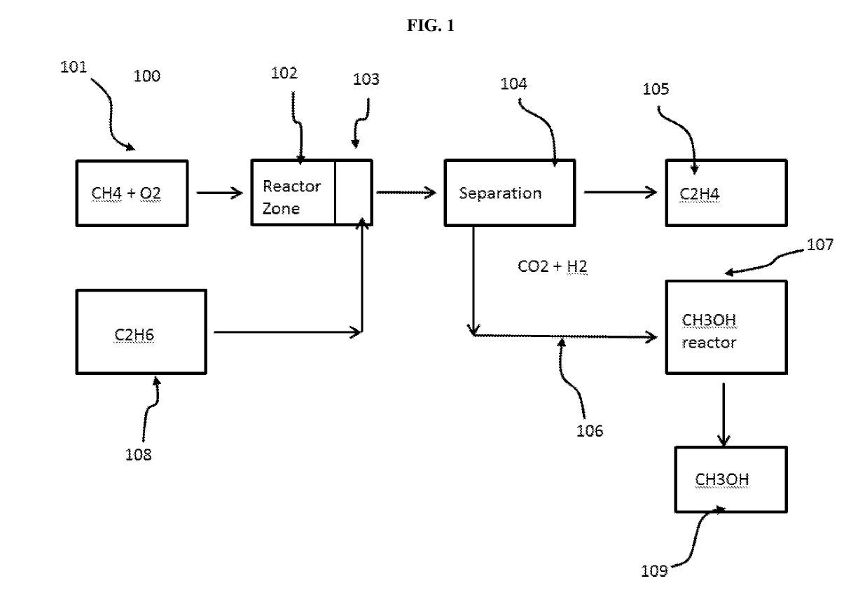

For the purpose of illustration and not limitation, FIG. 1 is a schematic representation of an exemplary system according to the disclosed subject matter. In certain embodiments, the system 100 can include two or more reactors 102 and 107. The methods of the present disclosure can involve reactors and reaction chambers suitable for reactions of hydrocarbon reactants and reagents catalyzed by solid catalysts. The reactor can be constructed of any suitable materials capable of holding high temperatures, for example from about 200° C. to about 1000° C. Non-limiting examples of such materials can include metals, alloys (including steel), glasses, ceramics or glass lined metals, and coated metals. The reactor can be a single reactor capable of withstanding oxidative catalytic cracking with a hydrocarbon feed. The reactor can be a single reactor with one or more zones. In certain embodiments, a reactor suitable for oxidative conversion of methane includes a post-reactor zone. In certain embodiments, additional streams, e.g., ethane, can be introduced to the post-reactor zone.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

In certain embodiments, the system 100 can include one or more feed lines 101 to introduce one or more reactants to a reactor 102, e.g., a reactor for oxidative conversion of methane. Non-limiting examples of the reactant include methane, oxygen and combinations thereof. In certain embodiments, the reactor 102 includes a post-reactor zone 103. Another feed line 108 can be coupled to the post-reactor zone 103 to introduce one more reactants. Non-limiting examples of the reactant include ethane. In certain embodiments, a post-reactor zone 103 can utilize the heat generated in reactor 102 to fuel endothermic reactions, e.g., dehydrogenation of ethane to ethylene.

In certain embodiments, reactor 102 is coupled to a separation unit 104. The separation unit 104 can be any type of separation unit known in the art. The separation unit 104 can include one or more transfer lines to transport separated products. In certain embodiments, a transfer line 105 can transport products including, but not limited to, ethylene. In certain embodiments, a transfer line 106 can transport products including, but not limited to, carbon dioxide and hydrogen.

In certain embodiments, a transfer line 106 can introduce products to a second reactor 107, e.g., a reactor for methanol synthesis. In certain embodiments, a transfer line 109 can transport products including, but not limited to, methanol. Alternatively, a second reactor 107 can be a reactor for syngas synthesis.

In certain embodiments, the pressure within a reaction chamber can be varied, as is known in the art. In certain embodiments, the pressure within a reaction chamber can be from about 1 psi to about 1000 psi. In certain embodiments, the pressure within a reaction chamber can be from about 250 psi to about 900 psi. In certain embodiments, the pressure within the reaction chamber can be from about 750 psi to about 800 psi.

Catalysts suitable for use in conjunction with the presently disclosed matter can be catalysts capable of catalyzing exothermic reactions of OCM and/or conversion of CO₂, and/or CO, to methanol. In certain embodiments, the first catalyst is capable of catalyzing the following reactions:

2CH₄+O₂→C₂H₄+2H₂O

2CH₄+½O₂→C₂H₆+H₂O

CH₄+1.5O₂→CO+2H₂O

CH₄+2O₂→CO₂+2H₂O (3)

In certain embodiments, the second catalyst is capable of catalyzing the following reaction:

CO₂+3H₂→CH₃OH+H₂O (4)

In certain embodiments, the total reaction of methane conversion can be summarized as follows:

3CH₄+2C₂H₆+3O₂→4C₂H₄+CH₃OH+5H₂O (5)

In certain embodiments, the catalysts can be solid catalysts, e.g., a solid-supported catalyst. The catalysts can be metal oxides or mixed metal oxides. In certain embodiments, the catalysts can be located in a fixed packed bed, i.e., a catalyst fixed bed. In certain embodiments, the catalysts can include solid pellets, granules, plates, tablets, or rings.

In certain embodiments, the first catalyst can include one or more transition metals or a mixture of alkali and alkali earth metal oxides. In certain embodiments the catalyst is modified with redox elements or alkaline chloride. The first catalyst can include nickel (Ni), sodium (Na), tungsten (W), and/or manganese (Mn). In certain embodiments, the first catalyst can include from about 1 to about 20% Na. In certain embodiments, the first catalyst can include about 10% Na. In certain embodiments, the first catalyst can include from about 1 to about 20% Mn. In certain embodiments, the first catalyst can include about 15% Mn. In certain embodiments, the first catalyst can include about 10% Na and about 15% Mn. In certain embodiments, the first catalyst can include about 3% Ni.

In certain embodiments, the second catalyst can include one or more transition metals. The second catalyst can include copper (Cu), zinc (Zn), Aluminum (Al), chromium (Cr), and/or zirconium (Zr). In certain embodiments, the second catalyst can include from about 40 to about 70% Cu. In certain embodiments, the second catalyst can include about 44.26%, 55.2%, or 69.3% Cu. In certain embodiments, the second catalyst can include from about 20 to about 40% Zn. In certain embodiments, the second catalyst can include about 27.4%, 36.44%, or 24.9% Zn. In certain embodiments, the second catalyst can include from about 1 to about 10% Cr. In certain embodiments, the second catalyst can include about 4.24% Cr. In certain embodiments, the second catalyst can include from about 5 to about 25% Zr. In certain embodiments, the second catalyst can include about 19.83% Zr.

In certain embodiments, the first or second catalyst can include a solid support. That is, the catalyst can be solid-supported. By way of non-limiting example, the solid support can constitute about 40%, 45%, 50%, 55%, 60%, 65%, 70%, 75%, 80%, 85%, 90%, or 95% of the total weight of the catalyst. In certain embodiments, the solid support can be MgO, La₂O₃, SiO₂ and/or Al₂O₃. In other embodiments, the first catalyst 10% Na-15% Mn/SiO₂, NaCl—Mn/SiO₂, Na₂WO₄—Mn/SiO₂ or 3% Ni/La₂O₃. In certain embodiments, the second catalyst is 69.3% CuO-27.4% ZnO-4.24% Cr₂O₃-3.97% Al₂O₃, 44.26% CuO-36.44% ZnO-11.68% Al₂O₃, or 55.2% CuO-24.9% ZnO-19.83% ZrO₂.

The catalysts of the presently disclosed subject matter can be prepared according to various techniques known in the art. For example, metal oxide catalysts suitable for use in catalyzing exothermic reactions of natural gas with oxygen and catalyzing reactions of CO₂ to form methanol, or reactions of CO₂ and/or CO to form syngas, can be prepared from various metal nitrates, metal halides, metal salts of organic acids, metal hydroxides, metal carbonates, metal oxyhalides, metal sulfates, and the like. In certain embodiments, a transition metal (e.g., Ni) can be precipitated along with a solid support (e.g., La₂O₃). In certain embodiments, catalysts can be prepared by precipitation of metal nitrates.

The presently disclosed subject matter also provides methods of conversion of methane to ethylene and methanol. In certain embodiments, the heat produced by methane oxidation is used to crack ethane and methanol is produced by conversion of carbon dioxide. In alternative embodiments, carbon dioxide can be converted to syngas.

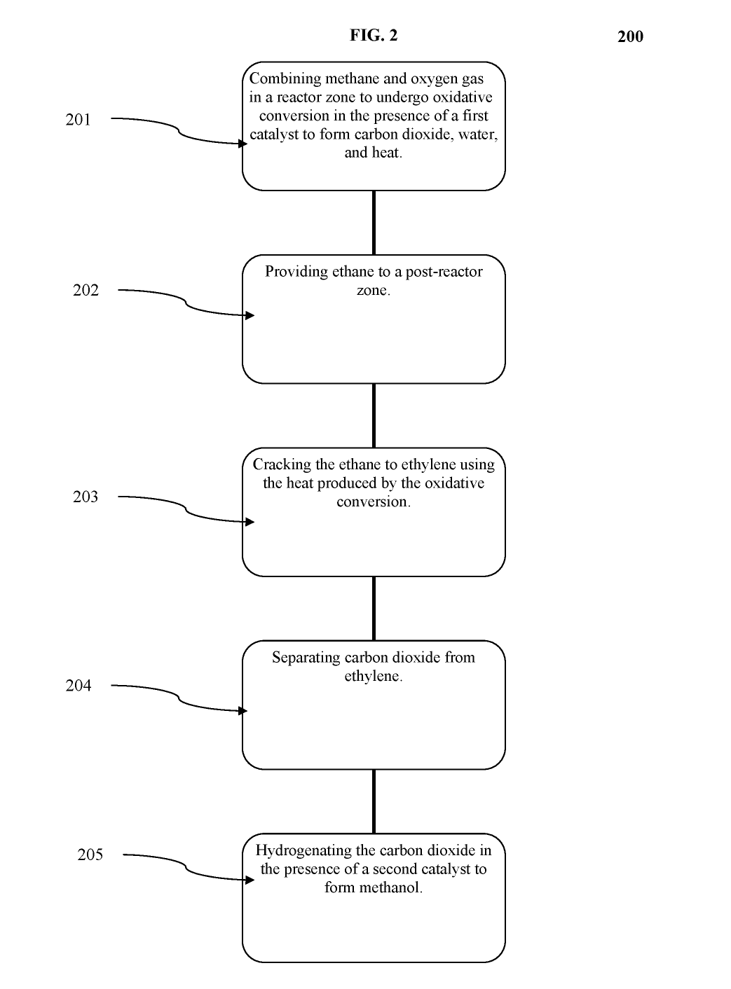

For the purpose of illustration and not limitation, FIG. 2 is a schematic representation of a method according to non-limiting embodiments of the disclosed subject matter. In certain embodiments and as shown in FIG. 2, the method 200 can include combining methane and oxygen gas in a reactor zone to undergo oxidative conversion in the presence of a first catalyst to form carbon dioxide, water, and heat 201.

In certain embodiments, oxygen can be a stream of pure O₂ and/or a stream of air which includes O₂. In certain embodiments, methane can be obtained from natural gas.

In certain embodiments, the method 200 can further include providing ethane to a post-reactor zone 202 and cracking the ethane to ethylene in situ using the heat produced by the oxidative conversion 203. In certain embodiments, the method can further include separating carbon dioxide from ethylene 204 to produce a stream of carbon dioxide. In certain embodiments, the method includes hydrogenating the carbon dioxide in the presence of a second catalyst to form methanol 205. In certain embodiments, carbon dioxide is hydrogenated in a second reactor. In certain embodiments, hydrogen gas is provided to the second reactor for the hydrogenation reaction. In alternative embodiments, carbon dioxide can be converted to syngas.

Reaction mixtures suitable for use with the presently disclosed methods can include various proportions of methane and oxygen. In certain embodiments, the reaction mixture can include a ratio of methane to oxygen of about 1 to about 5. In certain embodiments, the ratio is about 2.2. In certain embodiments, air is the source of oxygen.

The reaction temperature can be understood to be the temperature within the reaction chamber, i.e., for methane oxidative conversion or hydrogenation. In certain embodiments, the reaction temperature for methane oxidative conversion can be greater than 700° C., e.g., greater than about 710° C., 720° C., 730° C., 740° C., 750° C., 760° C., 780° C., or 790° C. In certain embodiments, the methane oxidative conversion reaction temperature can be from about 700° C. to about 900° C. or from about 750° C. to about 850° C. In certain embodiments, the methane oxidative conversion reaction temperature can be about 830° C., about 740° C., or about 720° C.

In certain embodiments, the reaction temperature for hydrogenation of CO₂ to methanol can be from about 200° C. to about 300° C. In certain embodiments, the reaction temperature for hydrogenation is about 250° C.

In certain embodiments, the reaction pressure can be about atmospheric pressure. In certain embodiments, the reaction pressure for hydrogenation of CO₂ can be from about 750 to about 800 psi. In certain embodiments, the pressure is about 750 psi or about 800 psi.

In alternative embodiments, carbon dioxide can be converted to syngas depending upon the specific reaction conditions and catalyst. For example, if the hydrogenation reaction temperature is high, e.g., 600° C. or more, it is possible to produce a syngas composition with high conversion of CO₂ but without methanol. In this case, the syngas can be converted to methanol through a second step where both CO and CO₂ can be converted to methanol. The conversion can proceed with partial conversion of CO₂ and H₂, thus providing a product mixture that includes CO, H₂O, CO₂, and H₂. The degree of conversion of CO₂ and H₂, as well as the ratio of CO₂ and H₂ in the reaction mixture, can influence the ratio of H₂ and CO in the syngas product formed. For example, use of a higher molar ratio of H₂:CO₂, for example 2:1 versus 1:1, in the reaction mixture can increase the molar ratio of H₂:CO in the product mixture. In certain embodiments, the molar ratio of H₂:CO₂, in the feed can vary from about 2 to about 3. A molar ratio of 1:1 is not suitable for methanol synthesis.

In certain embodiments, the product mixture can include less than about 14 to about 15% CO₂, by mole or less than about 14% CO₂, by mole. For example, the product mixture can include about 19%, 18%, 17%, 16%, 15%, 14%, 13%, 12%, 11%, 10%, 9%, 8% by mole. In certain embodiments, the product mixture can include about 13.9% CO₂ by mole. In certain embodiments, the product mixture can include about 14.2% or about 10.4% CO₂ by mole.

In certain embodiments, the selectivity for ethylene is from about 10 to about 75% mol. In certain embodiments the selectivity can be about 13.5%, 44.2%, or 63.5% mol.

In certain embodiments, the selectivity for methanol is from about 10 to about 50% mol. In certain embodiments the selectivity can be about 33.3, 38.2, or 33.4% mol.

The methods of the presently disclosed subject matter can have advantages over other techniques known in the art for ethylene synthesis. The presently disclosed subject matter includes the surprising discovery that the process integration and conversion of all carbon resources to useful chemicals results in a highly carbon efficient process.

As demonstrated in the Examples, the methods of the presently disclosed subject matter can provide ethylene.

### EXAMPLES

### Example 1—Conversion of Methane

In this Example, methane was converted in the presence of catalyst.

Methane was converted in the presence of 10% Na-15% Mn—O/SiO₂ catalyst at 830° C. and space velocity 7000 h⁻¹. The catalyst loading was 4 ml in a quartz reactor. The ratio of methane to oxygen was 2.2. Oxygen was sourced from air. The conversion of methane was 32.5% mol. The selectivity of the reaction is summarized in Table 1.

TABLE 1
Selectivity (% mol)

| Product | Selectivity |
|---|---|
| C₂H₄ | 44.2 |
| C₂H₆ | 18.0 |
| CO₂ | 31.4 |
| CO | 6.4 |

### Example 2—Conversion of Methane

In this Example, methane was converted in the presence of a pre-treated catalyst.

Methane was converted in the presence of 10% Na-15% Mn—O/SiO₂ catalyst at 740° C. and space velocity 7000 h⁻¹. The catalyst was pre-treated with a mixture of 3% HCl and N₂, at reaction conditions, within 30 minutes before the reaction. The catalyst loading was 4 ml in a quartz reactor. The ratio of methane to oxygen was 2.2. Oxygen was sourced from air. The conversion of methane was 42.0% mol. The selectivity of the reaction is summarized in Table 2.

TABLE 2
Selectivity (% mol)

| Product | Selectivity |
|---|---|
| C₂H₄ | 63.5 |
| C₂H₆ | 10.1 |
| CO₂ | 5.3 |
| CO | 20.1 |

Treatment of the catalyst with HCl resulted in outlet gas that contained more CO than CO₂.

### Example 3—Conversion of Methane with Addition of Ethane

In this Example, methane was converted in the presence of catalyst.

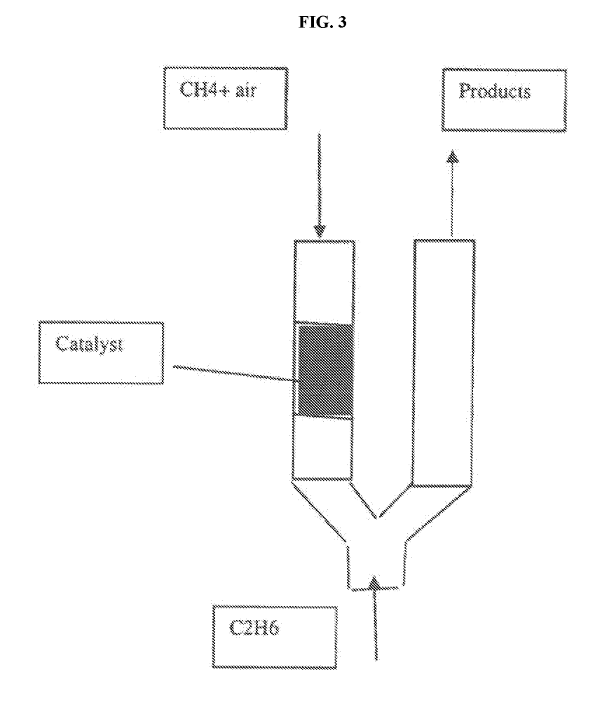

Methane was converted in the presence of 10% Na-15% Mn—O/SiO₂ catalyst at 830° C. and space velocity 7000 h⁻¹. The catalyst loading was 4 ml in a quartz reactor. The ratio of methane to oxygen was 2.2. Oxygen was sourced from air. Ethane was added to a post-reactor catalyst zone at 15% weight versus total methane and air. The reactor scheme is illustrated in FIG. 3. The conversion of methane was 34.2% mol. The selectivity of the reaction is summarized in Table 3.

TABLE 3
Selectivity (% mol)

| Product | Selectivity |
|---|---|
| C₂H₄ | 13.5 |
| C₂H₆ | 5 |
| CO₂ | 9 |
| CO | 3 |

### Example 4—Hydrogenation of CO₂

In this Example, CO₂ was converted to methanol.

CO₂ was converted to methanol in the presence of 69.3% CuO-27.4% ZnO-4.24% Cr₂O₃-3.97% Al₂O₃ catalyst at 250° C. and pressure of 750 psi. The catalyst loading was 1 ml. The flow rate of H₂ was 24.7 cc/min and CO₂ was 8.5 cc/min. The performance of catalyst was evaluated after 7 days. CO₂ conversion was 13.9% mol. Selectivity is summarized in Table 4.

TABLE 4
Selectivity (% mol)

| Product | Selectivity |
|---|---|
| CH₃OH | 33.3 |
| CO | 66.7 |

### Example 5—Hydrogenation of CO₂

In this Example, CO₂ was converted to methanol.

CO₂ was converted to methanol in the presence of 44.26% CuO-36.44% ZnO-11.68% Al₂O₃ catalyst at 250° C. and pressure of 800 psi. The catalyst loading was 1 ml. The flow rate of H₂ was 32 cc/min and CO₂ was 8.5 cc/min. The performance of catalyst was evaluated after 45 days. CO₂ conversion was 14.2% mol. Selectivity is summarized in Table 5.

TABLE 5
Selectivity (% mol)

| Product | Selectivity |
|---|---|
| CH₃OH | 38.2 |
| CO | 61.8 |

### Example 6—Hydrogenation of CO₂

In this Example, CO₂ was converted to methanol.

CO₂ was converted to methanol in the presence of 55.2% CuO-24.9% ZnO-19.83% ZrO₂ catalyst at 250° C. and pressure of 750 psi. The catalyst loading was 1 ml. The flow rate of H₂ was 124 cc/min and CO₂ was 42 cc/min. The performance of catalyst was evaluated after 120 days. CO₂ conversion was 10.4% mol. Selectivity is summarized in Table 6.

TABLE 6
Selectivity (% mol)

| Product | Selectivity |
|---|---|
| CH₃OH | 33.4 |
| CO | 66.6 |

### Example 7—Hydrogenation of CO₂

In this Example, CO₂ was converted to methanol with the addition of CO to the feed.

CO₂ was converted to methanol in the presence of catalyst 44.26% CuO-36.44% ZnO-11.68% Al₂O₃ at 250° C. and pressure of 750 psi. The catalyst loading was 1 ml. The flow rate of the total gas mixture was 130 cc/min. The gas mixture was 84.7% H₂, 1.85% CO₃ and 12.4% CO₂. The performance of catalyst was evaluated after 6 days. CO₂ conversion was 14% mol. Selectivity is summarized in Table 7.

TABLE 7
Selectivity (% mol)

| Product | Selectivity |
|---|---|
| CH₃OH | 33.4 |
| CO | 3.76 (concentration) |

The addition of CO to the hydrogenation feed increased the CO concentration in the products. This indicated that CO₂ conversion to methanol proceeded mostly through CO formation.

The method allowed hydrogenation of both deep oxidation products, such as CO and CO₂, to methanol. When the concentration of CO₂ was greater it required the application of more ethane to produce hydrogen for hydrogenation, but in the case when CO was the main product, hydrogen usage was reduced 34%.

### Example 8—Formation of Catalyst

The catalysts of Examples 1-7 were prepared as follows.

All the catalysts for CO₂ hydrogenation were prepared by co-precipitation of the elements from their nitrate salts by ammonium nitrate. The selected amount of the nitrates were mixed and dissolved in water and ammonium nitrate was gradually added to the solution to keep the pH of the solution pH=7. The precipitate was washed with water twice and then dried at 120° C. for 12 hours. The product was then calcined at 550° C. for 4 hours.

### Example 9—Conversion to Syngas

In this Example, CO₂ is converted to syngas by oxidative methane reforming.

A gas feed including methane was reacted in the presence of 0.5 ml 3% Ni/La₂O₃ catalyst at 720° C. The gas feed included 28.4% CH₄, 17.4% CO₂, 11% O₂, and 42.8% N₂. The catalyst was prepared by the co-precipitation method of Example 8.

The methane conversion was 72.7% mol. CO₂ conversion was 86.1% mol and the ratio of H₂ to CO was 1.5.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

1. A process for preparing ethylene from natural gas, the process comprising the steps of:
(a) combining methane and oxygen gas in a reactor zone to undergo oxidative conversion to form produced ethylene, carbon dioxide, water, and heat;
(b) providing ethane to a post-reactor zone;
(c) cracking the ethane using the heat produced by the oxidative conversion to form ethylene; and
(d) contacting the produced carbon dioxide with a first catalyst to generate methanol.

2. The process of claim 1, wherein the combining further includes contacting methane and oxygen gas with a second catalyst in the reactor zone.

3. The process of claim 2, wherein the second catalyst comprises 10% Na-15% Mn—O/SiO₂.

4. The process of claim 1, wherein the first catalyst comprises CuO—ZnO—Cr₂O₃—Al₂O₃.

5. The process of claim 4, wherein the first catalyst comprises 69.3% CuO-27.4% ZnO-4.24% Cr₂O₃-3.97% Al2O3.

6. The process of claim 1, wherein the first catalyst comprises CuO—ZnO—Al₂O₃.

7. The process of claim 6, wherein the first catalyst comprises 44.26% CuO-36.44% ZnO-11.68% Al₂O₃.

8. The process of claim 1, wherein the first catalyst comprises 55.2% CuO-24.9% ZnO-19.83% ZrO₂.

9. The process of claim 1, wherein the contacting proceeds at a pressure of from about 250 psi to about 900 psi.

10. The process of claim 9, wherein the pressure is from about 750 psi to about 800 psi.

11. The process of claim 1, wherein the combining proceeds at a temperature from about 750° C. to about 850° C.

12. The process of claim 11, wherein the temperature is about 830° C., about 740° C., or about 720° C.

13. The process of claim 1, wherein the contacting proceeds at a temperature from about 200° C. to about 300° C.

14. The process of claim 13, wherein the temperature is about 250° C.

15. The process of claim 1, wherein ethylene selectivity is from about 10 to about 75% mol.

16. The process of claim 15, wherein the selectivity is about 13.5%, 44.2%, or 63.5% mol.

17. A process for preparing ethylene from natural gas, the process comprising the steps of:
(a) combining methane and oxygen gas in a reactor zone to undergo oxidative conversion to form produced ethylene, carbon dioxide, water, and heat;
(b) providing ethane to a post-reactor zone;
(c) cracking the ethane using the heat produced by the oxidative conversion to form ethylene; and
(d) contacting the produced carbon dioxide with a catalyst to generate syngas.

18. The process of claim 17, wherein the catalyst comprises 3% Ni/La₂O₃.

19. The process of claim 17, wherein the combining further comprises O₂ and N₂.

20. The process of claim 19, wherein the combining further comprises 28.4% CH₄, 17.4% CO₂, 11% O₂, and 42.8% N₂.
